(19) United States
(12) Patent Application Publication
(10) Pub. No.: US 2017/0151154 A1
(43) Pub. Date: Jun. 1, 2017

(54) HAIR TREATMENT AGENTS

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/365,701

(22) Filed: Nov. 30, 2016

(30) Foreign Application Priority Data
Dec. 1, 2015 (DE) 10 2015 223 847.4

(51) Int. Cl. A61K 8/73; A61K 8/46; A61Q 5/00; A61K 8/64; A61Q 5/02; A61Q 5/12; A61K 8/34; A61K 8/42

(52) U.S. Cl. CPC A61K 8/737; A61K 8/347; A61K 8/466; A61K 8/42; A61K 8/731; A61K 8/64; A61Q 5/02; A61Q 5/12; A61Q 5/004; A61K 2800/5426; A61K 2800/596; A61K 2800/594

(57) ABSTRACT

Hair treatment agents including: an anionic surfactant from the group of alpha-olefin sulfonates, an anionic surfactant from the group of taurides, an amphoteric surfactant, a cationic polymer, and o-cymen-5-ol.

### BACKGROUND

The present invention relates to hair treatment agents. In particular, to shampoos and conditioners having active ingredients for hair care.

The importance of hair care products with the longer performance has grown. This is due in part to increased stress on hair, for example, from dyeing, permanent waves, cleaning of hair with shampoos, and due to environmental pollution. Such hair care products have an influence on the natural structure and properties of hair. For example, the wet and dry combability of hair, the hold and body of hair, and/or protection from increased split ends may be impacted by use of appropriate hair care products.

It has been customary to subject hair to special after-treatments in which the hair is treated with special active ingredients, for example, quaternary ammonium salts or special polymers. This is usually in the form of a rinse. These treatments may result in improved combability, hold, and body of hair while reducing the amount of split ends, depending on the formulation.

Multifunctional cosmetic products are also known in the prior art. In particular, this includes “two-in-one” shampoos, which clean and condition the hair. Such products are appreciated by consumers because the product eliminates the need for at least one procedural step, e.g., conditioning with a traditional hair conditioner.

### DETAILED DESCRIPTION

Similarly, products for modifying the natural hair color play a prominent role in hair cosmetics. Permanent, semipermanent, or temporary coloring systems are differentiated which are based on chemical and/or natural dyes. The hair colors produced artificially by permanent, semipermanent, or temporary coloring systems have the disadvantage, however, that they can change in an undesirable way, for example, during or after the cleaning of hair.

An “undesirable change” is understood to be the fading or bleeding and the loss of color brilliance of the hair shade achieved by the particular dyeing. Environmental effects and/or the effects of sunlight can intensify these changes still further.

The use of divalent metal salts in hair dyes to improve the durability and thereby the fastness of the color is known from EP 2438900 A1, which is incorporated by reference.

There continues to be a need to provide active substances or active substance combinations for hair treatment agents with good care properties, which, moreover, strengthen the adhesion of dyes to hair fibers and thus maintain the fastness of the artificially produced hair color, and in this respect to develop hair treatment agents further.

It has now been found that a combination of certain ingredients has an especially positive effect on dyed hair treated therewith and on the hair follicle.

A first subject of the present invention is hair treatment agents, including: at least one anionic surfactant from the group of alpha-olefin sulfonates, at least one anionic surfactant from the group of taurides, at least one amphoteric surfactant, at least one cationic polymer, and o-cymen-5-ol.

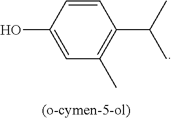

Hair treatment agents in the context of the present invention include: hair shampoos, hair conditioners, conditioning shampoos, hair sprays, hair rinses, hair treatments, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, and/or combinations thereof. As men in particular are often reluctant to use multiple different agents and/or multiple application steps, such agents are preferred that a man uses in any event. Preferred agents include: shampoos, conditioning agents, and/or hair tonics.

The hair treatment agents include at least one anionic surfactant from the group of alpha-olefin sulfonates. Preferably, olefin sulfonates are obtained by the addition of SO₃ to olefins of the formula R²—CH═CH—R³, where R² and R³ independently of one another stand for H or alkyl groups having 1 to 20 carbon atoms, with the proviso, that R² and R³ together have at least 6 and preferably 10 to 16 carbon atoms.

alpha-Olefin sulfonates arising when R² or R³ stands for hydrogen are used according to the invention. Typical examples of olefin sulfonates that are used are the sulfonation products obtained by reacting SO₃ with 1-, 2-butene, 1-, 2-, 3-hexene, 1-, 2-, 3-, 4-octene, 1-, 2-, 3-, 4-, 5-decene, 1-, 2-, 3-, 4-, 5-, 6-dodecene, 1-, 2-, 3-, 4-, 5-, 6-, 7-tetradecene, 1-, 2-, 3-, 4-, 5-, 6-, 7-, 8-hexadecene, 1-, 2-, 3-, 4-, 5-, 6-, 7-, 8-, 9-octadecene, 1-, 2-, 3-, 4-, 5-, 6-, 7-, 8-, 9-, 10-eicosene, and 1-, 2-, 3-, 4-, 5-, 6-, 7-, 8-, 9-, 10-, and 11-docosene. A neutralization is carried out after a completed sulfonation, after which the olefin sulfonate is present in the mixture as an alkali, alkaline earth, ammonium, alkylammonium, alkanolammonium, glucammonium, and preferably sodium salt. Olefin sulfonates can be used both in aqueous pastes, preferably at a pH of 7 to 10, as well as water-free products, preferably as granules, as are obtained by conventional spray drying, drying in the thin-layer evaporator (“flash dryer”), or in a fluidized bed dryer.

Hair treatment agents preferred according to the invention include, based on their weight, 0.5 to 20% by weight, preferably 0.75 to 15% by weight, more preferably 1 to 12% by weight, and in particular 2 to 10% by weight of anionic surfactant(s) from the group of alpha-olefin sulfonates.

Hair treatment agents particularly preferred according to the invention include, based on their weight, 0.5 to 20% by weight, preferably 0.75 to 15% by weight, more preferably 1 to 12% by weight, and in particular 2 to 10% by weight of reaction products of C₁₂-C₁₈ olefins with sulfur trioxide, which consist substantially of alkene sulfonates and hydroxyalkane sulfonates.

The hair treatment agents include at least one anionic surfactant from the group of taurides.

Taurides, also N-acyltaurides, N-methyl-N-acyltaurates, N-acyltaurates, or N-acyltaurines, are a group of mild anionic surfactants. Their hydrophilic head group consists of N-methyltaurine (2-methylaminoethanesulfonic acid), and the lipophilic group consists of a long-chain carboxylic acid (fatty acid) linked via an amide bond. Lauric (C₁₂), myristyc (C₁₄), palmitic (C₁₆), and stearic acid (C₁₈) are used as fatty acids, but primarily oleic acid (C_{18:1}) and coconut fatty acid mixture (C₈-C₁₈).

The general structural formula for taurides is:

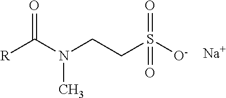

Hair treatment agents preferred according to the invention include, based on their weight, 0.3 to 10% by weight, preferably 0.5 to 8% by weight, more preferably 0.6 to 7% by weight, and in particular 0.7 to 5% by weight of taurides of the formula (I)

R¹CON(CH₃)CH₂CH₂SO₃X (I),

where: R¹ is a linear or branched alkyl group having 6 to 30, preferably 8 to 22 C atoms, or a linear or branched mono- or polyunsaturated alkenyl group having 6 to 30, preferably 8 to 22 C atoms, and X is a counterion, preferably an alkali metal, alkaline earth metal, or ammonium ion.

Preferably R¹ in formula (I) stands for a cocoyl group (C₈-C₁₈ coconut fatty acid mixture). Hair treatment agents particularly preferred according to the invention include, based on their weight, 0.3 to 10% by weight, preferably 0.5 to 8% by weight, more preferably 0.6 to 7% by weight, and in particular 0.7 to 5% by weight of sodium methyl cocoyl taurate.

Suitable surfactants are marketed, for example, under the trade names: Adinol®, Geropon®, Hostapon®, Metaupon®, Nikkol@, Protapon®, Pureact®, and Tauranol®.

It has emerged as preferable to minimize the amount of sulfate surfactants in the hair treatment agents of the invention in order to make them even milder and more effective. Hair treatment agents preferred according to the invention are characterized in that they include, based on their weight, less than 1% by weight, preferably less than 0.5% by weight, more preferably less than 0.5% by weight, and in particular less than 0.1% by weight of sulfate surfactant(s).

The hair treatment agents of the invention include at least one anionic amphoteric surfactant. Surfactants that have both a negatively and also a positively charged functional group are called amphoteric surfactants, also known as zwitterionic surfactants.

Particularly suitable zwitterionic surfactants are the so-called betaines, such as the N-alkyl-N,N-dimethylammonium glycinates, for example, coco alkyl dimethylammonium glycinate and N-acylaminopropyl-N,N-dimethylammonium glycinates, for example, cocoacylaminopropyl dimethylammonium glycinate, and 2-alkyl-3-carboxymethyl-3-hydroxyethyl imidazolines each having 8 to 18 carbon atoms in the alkyl or acyl group, as well as coco acylaminoethyl hydroxyethyl carboxymethyl glycinate. A preferred zwitterionic surfactant is the fatty acid amide derivative known under the INCI name of Cocamidopropyl Betaine.

Further examples of suitable ampholytic surfactants are N-alkylglycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids each having about 8 to 24 carbon atoms in the alkyl group.

Particularly preferred ampholytic surfactants are N-cocoalkylaminopropionate, cocoacylaminoethylaminopropionate, and C₁₂-C₁₈ acylsarcosine.

Preferred hair treatment agents of the invention include, based on their weight, 0.3 to 10% by weight, preferably 0.5 to 8% by weight, more preferably 0.75 to 6% by weight, and in particular 1 to 5% by weight of amphoteric surfactant(s).

Particularly preferred hair treatment agents of the invention are characterized in that they include amphoteric surfactant(s) from the groups of: N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids each having about 8 to 24 C atoms in the alkyl group, alkylaminoacetic acids each having about 8 to 24 C atoms in the alkyl group, N-cocoalkylaminopropionate, cocoacylaminoethylaminopropionate, C₁₂-C₁₈ acylsarcosine, N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, 2-alkyl-3-carboxymethyl-3-hydroxyethyl imidazolines each having 8 to 18 C atoms in the alkyl or acyl group, cocoacylaminoethylhydroxyethylcarboxymethyl glycinate, the compounds known under the INCI name Cocamidopropyl Betaine, and/or the compounds known under the INCI name Disodium Cocoamphodiacetate.

Preferred agents include the amphoteric surfactant(s) in amounts of 0.3 to 10% by weight, preferably 0.5 to 8% by weight, more preferably 0.75 to 6% by weight, and in particular 1 to 5% by weight, based in each case on the weight of the total agent.

Particularly preferred hair treatment agents include as amphoteric surfactants betaines of the formula (Bet-I)

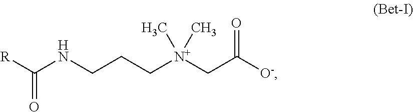

in which R stands for a straight-chain or branched, saturated or mono- or polyunsaturated alkyl or alkenyl group having 8 to 24 carbon atoms.

These surfactants are called Amidopropylbetaines according to the INCI nomenclature, the representatives, derived from coconut fatty acids, being preferred and being called cocoamidopropyl betaines. Used with particular preference according to the invention are surfactants of the formula (Bet-I), which are a mixture of the following representatives:
H₃C—(CH₂)₇—C(O)—NH—(CH₂)₃N⁺(CH₃)₂CH₂COO⁻,
H₃C—(CH₂)₉—C(O)—NH—(CH₂)₃N⁺(CH₃)₂CH₂COO⁻,
H₃C—(CH₂)₁₁—C(O)—NH—(CH₂)₃N⁺(CH₃)₂CH₂COO⁻,
H₃C—(CH₂)₁₃—C(O)—NH—(CH₂)₃N⁺(CH₃)₂CH₂COO⁻,
H₃C—(CH₂)₁₅—C(O)—NH—(CH₂)₃N⁺(CH₃)₂CH₂COO⁻, and/or
H₃C—(CH₂)₇—CH═CH—(CH₂)₇—C(O)—NH—(CH₂)₃N⁺(CH₃)₂CH₂COO⁻.

Used with particular preference are surfactants of the formula (Bet-I) within rather narrow amount ranges. Here hair treatment agents of the invention are preferred, which include, based on their weight, 0.25 to 8% by weight, more preferably 0.5 to 7% by weight, more preferably 0.75 to 6.5% by weight, and in particular 1 to 5.5% by weight of surfactant(s) of the formula (Bet-I).

In addition to amphoteric surfactant(s) of the formula (Bet-I) or in their place, the hair treatment agents of the invention can include with particular preference as amphoteric surfactants betaines of the formula (Bet-II)

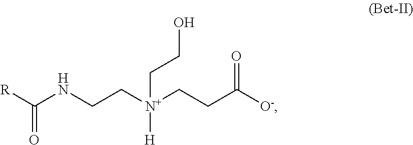

in which R stands for a straight-chain or branched, saturated or mono- or polyunsaturated alkyl or alkenyl group having 8 to 24 carbon atoms.

These surfactants are called Amphoacetates according to the INCI nomenclature, the representatives, deriving from coconut fatty acids, being preferred and being called Cocoamphoacetates.

For production-related reasons, surfactants of this type always also include betaines of the formula (Bet-IIa)

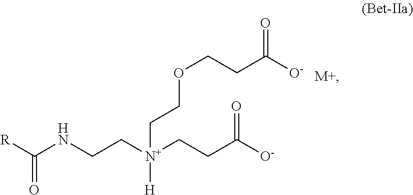

in which R stands for a straight-chain or branched, saturated or mono- or polyunsaturated alkyl or alkenyl group having 8 to 24 carbon atoms and M for a cation.

These surfactants are called Amphodiacetates according to the INCI nomenclature, the representatives, deriving from coconut fatty acids, being preferred and being called Cocoamphodiacetates.

Particularly preferred according to the invention are surfactants of the formula (Bet-II), which are a mixture of the following representatives:
H₃C—(CH₂)₇—C(O)—NH—(CH₂)₂NH⁺(CH₂CH₂OH)CH₂CH₂COO⁻,
H₃C—(CH₂)₉—C(O)—NH—(CH₂)₂NH⁺(CH₂CH₂OH)CH₂CH₂COO⁻,
H₃C—(CH₂)₁₁—C(O)—NH—(CH₂)₂NH⁺(CH₂CH₂OH)CH₂CH₂COO⁻,
H₃C—(CH₂)₁₃—C(O)—NH—(CH₂)₂NH(CH₂CH₂OH)CH₂CH₂COO⁻,
H₃C—(CH₂)₁₅—C(O)—NH—(CH₂)₂NH⁺(CH₂CH₂OH)CH₂CH₂COO⁻, and/or
H₃C—(CH₂)₇—CH═CH—(CH₂)₇—C(O)—NH—
(CH₂)₂NH⁺(CH₂CH₂OH)CH₂CH₂COO⁻.

Used with particular preference are surfactants of the formula (Bet-II) within rather narrow amount ranges. Here hair treatment agents of the invention are preferred, which include, based on their weight, 0.25 to 8% by weight, more preferably 0.5 to 7% by weight, more preferably 0.75 to 6.5% by weight, and in particular 1 to 5.5% by weight of surfactant(s) of the formula (Bet-II).

In summary, cosmetic agents of the invention are preferred in which the R group in the formulas (Bet-I) and (Bet-II) is selected from: H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, and/or mixtures thereof.

The hair treatment agents can include in addition nonionic surfactant(s) and/or cationic surfactant(s).

Suitable nonionic surfactants include, for example: adducts of 4 to 30 mol of ethylene oxide and/or 0 to 5 mol of propylene oxide to linear fatty alcohols having 8 to 22 C atoms, to fatty acids having 12 to 22 C atoms and to alkyl phenols having 8 to 15 C atoms in the alkyl group; ethylene oxide and polyglycerol adducts to methylglucoside fatty acid esters, fatty acid alkanolamides, and fatty acid glucamides; C₈-C₃₀ fatty acid monoesters and diesters of adducts of 1 to 30 mol of ethylene oxide to glycerol; amine oxides, sorbitan fatty acid esters and adducts of ethylene oxide to sorbitan fatty acid esters such as, for example, polysorbates; fatty acid alkanolamides of the following general formula,

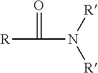

in which R preferably denotes a linear or branched, saturated or unsaturated alkyl or alkenyl group having 8 to 24 carbon atoms and the R′ groups stand for hydrogen or for the group —(CH₂)ₙOH, in which n denotes the numbers 2 or 3, with the proviso that at least one of the R′ groups stands for the aforementioned group —(CH₂)ₙOH; sugar fatty acid esters and adducts of ethylene oxide to sugar fatty acid esters; adducts of ethylene oxide to fatty acid alkanolamides and fatty amines; alkyl (oligo)glucosides; mixtures of alkyl (oligo)glucosides and fatty alcohols, for example, the commercially available product Montanove 68, adducts of 5 to 60 mol of ethylene oxide to castor oil and hydrogenated castor oil; partial esters of polyols having 3 to 6 carbon atoms with saturated fatty acids having 8 to 22 carbon atoms; sterols, where are understood to be a group of steroids that have a hydroxyl group at C atom 3 of the steroid skeleton, and are isolated from both animal tissue (zoosterols) and vegetable fats (phytosterols). Examples of zoosterols are cholesterol and lanosterol. Examples of suitable phytosterols are ergosterol, stigmasterol, and sitosterol. Sterols, the so-called mycosterols, are also isolated from fungi and yeasts; and/or phospholipids, for example, glucose phospholipids, which are obtained, e.g., as lecithins or phosphatidylcholines from, e.g., egg yolk or plant seeds (e.g., soybeans).

Suitable alkyl (oligo)glycosides can be selected from compounds of the general formula RO-[G]ₓ, in which [G] is derived preferably from aldoses and/or ketoses having 5-6 carbon atoms, preferably from glucose. The subscript x stands for the degree of oligomerization (DP), i.e., for the distribution of the mono- and oligoglycosides. The subscript x preferably has a value in the range of 1 to 10, particularly preferably in the range of 1 to 3; this value need not be an integer but may be a fraction, which can be determined analytically.

Particularly preferred alkyl (oligo)glycosides have a degree of oligomerization between 1.2 and 1.5. The R group preferably stands for at least one alkyl and/or alkenyl group having 4 to 24 carbon atoms.

Particularly preferred alkyl (oligo)glycosides are the compounds known under the INCI names Caprylyl/Capryl Glucoside, Decyl Glucoside, Lauryl Glucoside, and Coco Glucoside.

Suitable amine oxides can be selected from at least one compound of the general formula (A-I) or (A-II)

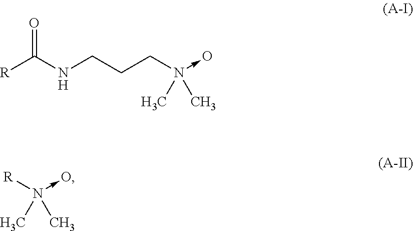

in which R in each case stands for a straight-chain or branched, saturated or mono- or polyunsaturated alkyl or alkenyl group having 6 to 24 carbon atoms, preferably having 8 to 18 carbon atoms.

The surfactants of the aforementioned formula (A-I) or (A-II), obtainable commercially from various suppliers and known under the INCI names Cocamine Oxide, Lauramine Oxide, and/or Cocamidopropyl Amine Oxide, are preferred in particular.

Of the suitable C₈-C₃₀ fatty acid mono- and diesters of adducts of 1 to 30 mol of ethylene oxide to glycerol, understood as preferred are those known under the INCI names PEG(1-10) Glyceryl Cocoate, in particular PEG-7 Glyceryl Cocoate.

It can be advantageous, furthermore, to combine the ethoxylated fatty acid esters with other ethoxylated fatty acid esters. Such product mixtures are commercially available, for example, under the name “Antil 200®” (INCI name: PEG-200 Hydrogenated Glyceryl Palmate, PEG-7 Glyceryl Cocoate) from the company Evonik.

Particularly preferred nonionic surfactants, which may be present in the hair treatment agents of the invention, are fatty acid alkanolamides, in particular the compounds known under the INCI names Cocamide MEA and/or Cocamide MIPA; alkyl (oligo)glucosides, in particular the compounds known under the INCI names Caprylyl/Capryl Glucoside, Decyl Glucoside, Lauryl Glucoside, and/or Coco Glucoside; C₈-C₃₀ fatty acid monoesters and diesters of adducts of 1 to 30 mol of ethylene oxide to glycerol, in particular the compound known under the INCI name PEG-7 Glyceryl Cocoate; and/or adducts of 4 to 30 mol of ethylene oxide and/or 0 to 5 mol of propylene oxide to linear fatty alcohols having 8 to 22 C atoms. Cocamide MEA and/or PEG-7 Glyceryl Cocoate are particularly preferred because of their foam-stabilizing and refatting properties.

Cationic surfactants of the type of quaternary ammonium compounds, esterquats, and amidoamines are also usable according to the invention. Preferred quaternary ammonium compounds are ammonium halides, particularly chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, such as, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred cationic surfactants are the imidazolium compounds known under the INCI names Quaternium-27 and Quaternium-83.

Particularly preferred hair treatment agents of the invention are characterized in that they include as a cationic care substance, based on their weight, 0.05 to 7.5% by weight, preferably 0.1 to 5% by weight, particularly preferably 0.2 to 3.5% by weight, and in particular 0.25 to 2.5% by weight of cationic surfactant(s) from the group of quaternary ammonium compounds and/or esterquats and/or amidoamines, preferred cationic surfactant(s) being selected from: alkyltrimethylammonium chlorides having preferably 10 to 18 carbon atoms in the alkyl group; dialkyldimethylammonium chlorides having preferably 10 to 18 carbon atoms in the alkyl group; trialkylmethylammonium chlorides having preferably 10 to 18 carbon atoms in the alkyl group; cetyltrimethylammonium chloride; stearyltrimethylammonium chloride; distearyldimethylammonium chloride; lauryldimethylammonium chloride; lauryldimethylbenzylammonium chloride; tricetylmethylammonium chloride; Quaternium-27; Quaternium-83; N-methyl-N(2-hydroxyethyl)-N,N-(ditalgacyloxyethyl)ammonium methosulfate; N-methyl-N(2-hydroxyethyl)-N,N-(distearoyloxyethyl)ammonium methosulfate; N,N-dimethyl-N,N-distearoyloxyethylammonium chloride; and/or N,N-di-(2-hydroxyethyl)-N,N-(fatty acid ester ethyl)ammonium chloride.

The agents of the invention include at least one cationic polymer.

Irrespective of which cationic polymer(s) are used, preferred hair treatment agents include, based on the weight of the agent, 0.01 to 3% by weight, preferably 0.05 to 2% by weight, more preferably 0.1 to 1.5% by weight, and in particular 0.15 to 0.8% by weight of cationic polymer(s).

Cationic polymers preferably usable according to the invention are described hereafter:

Homopolymers of the general formula (G1-I),

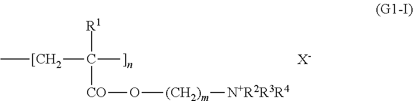

in which R¹=—H or is —CH₃, R², R³, and R⁴ independently of one another are selected from C1-4 alkyl, alkenyl, or hydroxyalkyl groups, m=1, 2, 3, or 4, n is a natural number and X is a physiologically acceptable organic or inorganic anion, and copolymers, consisting substantially of the monomer units listed in formula (G1-I) and nonionogenic monomer units, are particularly preferred cationic polymers. In the context of these polymers, those are preferred according to the invention for which at least one of the following conditions applies: R¹ stands for a methyl group; R², R³, and R⁴ stand for methyl groups; and/or m has the value of 2.

Suitable physiologically acceptable counterions X⁻ are, for example, halide ions, sulfate ions, phosphate ions, methosulfate ions, as well as organic ions such as lactate, citrate, tartrate, and acetate ions. Halide ions, in particular chloride, are preferred.

A particularly suitable homopolymer is the optionally crosslinked poly(methacryloyloxyethyl trimethylammonium chloride) with the INCI name Polyquaternium-37. Such products are commercially available, for example, under the names Rheocare® CTH (Cosmetic Rheologies) and Synthalene CR (Ethnichem). The crosslinking may occur, if desired, with the aid of olefinically polyunsaturated compounds, for example, divinylbenzene, tetraallyloxyethane, methylene bisacrylamide, diallyl ether, polyallyl polyglyceryl ether, or allyl ethers of sugars, or sugar derivatives such as erythritol, pentaerythritol, arabitol, mannitol, sorbitol, sucrose, or glucose. Methylene bisacrylamide is a preferred crosslinking agent.

The homopolymer is preferably used in the form of a nonaqueous polymer dispersion that should have a polymer proportion not less than 30% by weight. Such polymer dispersions are obtainable commercially under the names Salcare® SC 95 (approximately 50% polymer proportion, further components: mineral oil (INCI name: Mineral Oil) and tridecyl polyoxypropylene polyoxyethylene ether (INCI name: PPG-1 Trideceth-6)), and Salcare® SC 96 (approximately 50% polymer proportion, further components: mixture of diesters of propylene glycol with a mixture of caprylic and capric acid (INCI name: Propylene Glycol Dicaprylate/Dicaprate), and tridecyl polyoxypropylene polyoxyethylene ether (INCI name: PPG-1 Trideceth-6)).

Copolymers with monomer units according to formula (G1-I) include as nonionogenic monomer units preferably acrylamide, methacrylamide, acrylic acid-C₁₋₄-alkyl esters, and methacrylic acid-C₁₋₄-alkyl esters. Acrylamide is particularly preferable of these nonionogenic monomers. These copolymers as well can be crosslinked, as described above in the case of the homopolymers. A copolymer preferred according to the invention is the crosslinked acrylamide-methacryloyloxyethyltrimethylammonium chloride copolymer. Such copolymers, in which the monomers are present in a weight ratio of about 20:80, are available commercially as about 50% nonaqueous polymer dispersion under the name Salcare® SC 92.

Other preferred cationic polymers are, for example: quaternized cellulose derivatives, as are commercially available under the names Celquat® and polymer JR®. The compounds Celquat® H 100, Celquat® L 200, and polymer JR®400 are preferred quaternized cellulose derivatives; cationic alkyl polyglycosides; cationized honey, for example, the commercial product Honeyquat® 50; cationic guar derivatives, such as in particular the products sold under the trade names Cosmedia® Guar and Jaguar; polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers; copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755; vinylpyrrolidone-vinylimidazolium methochloride copolymers, as they are sold under the names Luviquat® FC 370, FC 550, FC 905, and HM 552; quaternized polyvinyl alcohol; and/or polymers having quaternary nitrogen atoms in the main polymer chain, known under the names Polyquaternium-2, Polyquaternium-17, Polyquaternium-18, and Polyquaternium-27.

The polymers known under the names Polyquaternium-24 (commercial product, e.g., Quatrisoft® LM 200) can likewise be used as cationic polymers. Also usable according to the invention are the copolymers of vinylpyrrolidone, as they are available as the commercial products Copolymer 845 (manufacturer: ISP), Gaffix® VC 713 (manufacturer: ISP), GafquatP ASCP 1011, Gafquat® HS 110, Luviquat® 8155, and Luviquat® MS 370.

Cationic protein hydrolysates can also be used as cationic polymers, preferred agents including one or more cationic protein hydrolysates from the group of Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, and Quaternium-79 Hydrolyzed Wheat Protein.

Cationic polysaccharide polymers according to the invention are used with particular preference as cationic polymers.

Cationic polysaccharide polymers increase the care performance of the hair treatment agents of the invention (in particular the effectiveness of the agents of the invention against hair breakage). Suitable cationic polysaccharide polymers can be selected from cationic cellulose compounds and/or from cationic guar derivatives.

Particularly preferred hair treatment agents of the invention include as cationic polysaccharide polymer(s), based on the weight of the agent, 0.01 to 3% by weight, preferably 0.05 to 2% by weight, more preferably 0.1 to 1.5% by weight, and in particular 0.15 to 0.8% by weight of at least one polymer from the group of cationic cellulose polymers and/or cationic guar derivatives.

Cationic cellulose compounds in the context of the invention are those that bear more than one permanent cationic charge in at least one side chain. Cellulose is made up of beta-1,4-glycosidically linked D-glucopyranose units and forms unbranched, water-insoluble chains. Chemical substituents that bond to the cellulose backbone and are not found in native cellulose, because they are introduced subsequently, e.g., by chemical synthesis, are defined as a “side chain” of a cellulose.

Preferred are quaternized cellulose polymers that result from hydroxy(C₂-C₄)alkyl celluloses, particularly preferably from hydroxyethyl celluloses.

Such polymers are known to the skilled artisan and available commercially from various companies. Particularly preferred are the cationic cellulose derivatives known under the INCI names Polyquaternium-4, Polyquaternium-10, Polyquaternium-24, Polyquaternium-67, and/or Polyquaternium-72. Very particularly preferred are Polyquaternium-10, Polyquaternium-24, and/or Polyquaternium-67, and Polyquaternium-10 is preferred in particular.

Preferred hair treatment agents of the invention include as cationic polysaccharide polymer(s), based on the weight of the agent, 0.01 to 3% by weight, preferably 0.05 to 2% by weight, more preferably 0.1 to 1.5% by weight, and in particular 0.15 to 0.8% by weight of at least one polymer from the group of Polyquaternium-4, Polyquaternium-10, Polyquaternium-24, Polyquaternium-67, and/or Polyquaternium-72.

Particularly preferred hair treatment agents of the invention include as cationic polysaccharide polymer(s), based on the weight of the agent, 0.01 to 3% by weight, preferably 0.05 to 2% by weight, more preferably 0.1 to 1.5% by weight, and in particular 0.15 to 0.8% by weight of Polyquaternium-10.

Suitable cationic guar derivatives in the context of the invention are cationic hydroxyalkyl guar derivatives, preferably cationic hydroxyethyltrimethylammonium guar and/or cationic hydroxypropyltrimethylammonium guar with average molecular weights between 100,000 and 2,000,000 Da. Particularly preferred are cationic guar polymers, known under the INCI name Guar Hydroxypropyltrimonium Chloride, with a molecular weight (weight average) between 200,000 and 1,600,000 Da. The cationic charge density of said guar polymers is preferably at least 0.4 mEq/g, preferably at least 0.5 mEq/g, and especially at least 0.6 mEq/g. Their nitrogen content is preferably within the range from 1.1 to 1.8% by weight (based on their total weight).

Cationic guar derivatives, which are known under the INCI name Guar Hydroxypropyltrimonium Chloride, are known to the skilled artisan and obtainable, for example, under the trade names Cosmedia® Guar, N-Hance®, and/or Jaguar® from various suppliers.

Particularly preferred hair treatment agents of the invention include as cationic polysaccharide polymer(s), based on the weight of the agent, 0.01 to 3% by weight, preferably 0.05 to 2% by weight, more preferably 0.1 to 1.5% by weight, and in particular 0.15 to 0.8% by weight of guar hydroxypropyltrimonium chloride.

The hair treatment agents of the invention include o-cymen-5-ol. This compound is also designated as cymenol or 4-isopropyl-m-cresol and is preferably used within specific narrow ranges. Hair treatment agents preferred according to the invention include, based on their weight, 0.001 to 0.4% by weight, preferably 0.01 to 0.3% by weight, more preferably 0.025 to 0.2% by weight, and in particular 0.05 to 0.1% by weight of o-cymen-5-ol.

The hair treatment agents include the above-described active substances preferably in a cosmetic acceptable carrier. In the context of the invention, this is understood to be preferably an aqueous or aqueous-alcoholic carrier.

The cosmetic carrier includes preferably at least 50% by weight, more preferably at least 60% by weight, particularly preferably at least 70% by weight, and especially preferably at least 75% by weight of water. Furthermore, the cosmetic carrier may include 0.01 to 40% by weight, preferably 0.05 to 30% by weight, and in particular 0.1 to 20% by weight of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of said alcohols.

The water-soluble alcohols are particularly preferred. Ethanol, 1,2-propylene glycol, glycerol, benzyl alcohol, and mixtures of said alcohols are particularly preferred.

It is of advantage for a very good (scalp) skin compatibility of the hair treatment agents of the invention if these have a slightly acidic pH. It has been found that the agents of the invention have an especially good skin compatibility and mildness in a pH range of 4.2 to 5.8.

In one example, the hair treatment agents of the invention preferably have a pH in the range of 4.2 to 5.8, more preferably from 4.25 to 5.6, particularly preferably from 4.3 to 5.5, exceedingly preferably from 4.35 to 5.4, and especially preferably from 4.4 to 5.3.

The hair treatment agents of the invention may include vegetable oils, vegetable butters, and/or vegetable waxes. These vegetable oil components give the hair an improved combability and stylability and increase the hair shine.

Suitable vegetable oil components include natural (vegetable) oils and/or butter, which typically include triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Camauba wax, beeswax, and/or candelilla wax can be used with preference as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are particularly preferred.

In a preferred embodiment, the hair treatment agents of the invention preferably include coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kemel oil, mango kemel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is particularly preferable if the hair treatment agents of the invention include (sweet) almond oil, peach kernel oil, apricot kernel oil, amaranth seed oil, olive oil, jojoba oil, cocoa butter, and/or shea butter.

The weight proportion of the at least one vegetable oil, vegetable butter, and/or vegetable wax in terms of the total weight of the hair treatment agents of the invention is preferably 0.02 to 2.50% by weight, more preferably of 0.03 to 2.00% by weight, particularly preferably 0.04 to 1.50% by weight, and in particular 0.05 to 1.00% by weight.

Apart from the aforementioned essential and facultative components, the hair treatment agents of the invention in a further preferred embodiment for a further increase in the care properties of the agents may include at least one further active hair-conditioning substance, which can be selected from the group of: protein hydrolysates, vitamins, plant extracts, and/or glycerol.

Suitable protein hydrolysates are understood to be product mixtures that can be obtained by acid-, base-, or enzyme-catalyzed degradation of proteins. Protein hydrolysates of plant, animal, and/or marine origin can be used.

Animal protein hydrolysates are, for example, elastin, collagen, keratin, silk, and milk protein hydrolysates, which can also be present in the form of salts. Such products are marketed, for example, under the trademarks Dehylan® (Cognis), Promois® (Interorgana), Collapurone (Cognis), Nutrilan®(Cognis), Gelita-Sol® (Deutsche Gelatine Fabriken Stoess & Co), Lexein®(Inolex), and Kerasol® (Croda).

Preferred are protein hydrolysates of plant origin, e.g., soybean, almond, rice, pea, potato, and wheat protein hydrolysates. Such products are available, for example, under the tradenames Gluadin® (Cognis), DiaMin®(Diamalt), Lexein® (Inolex), and Crotein® (Croda). Cationized protein hydrolysates can also be used, wherein the underlying protein hydrolysate may originate from animals, for example, from collagen, milk, or keratin, from plants, for example, from wheat, corn, rice, potatoes, soybeans, or almonds, from marine life forms, for example, from fish collagen or algae, or from biotechnologically obtained protein hydrolysates. The protein hydrolysates underlying the cationic derivatives may be obtained from the corresponding proteins by chemical, in particular, alkaline or acidic hydrolysis, by enzymatic hydrolysis, and/or by a combination of both types of hydrolysis. The hydrolysis of proteins usually gives rise to a protein hydrolysate with a molecular weight distribution of about 100 Da up to several thousand daltons. Preferred cationic protein hydrolysates are those whose underlying protein portion has a molecular weight of 100 up to 25,000 Da, preferably 250 to 5000 Da. Furthermore, cationic protein hydrolysates are understood to be quaternized amino acids and mixtures thereof. Quaternization of the protein hydrolysates or of the amino acids is often performed by means of quaternary ammonium salts such as, for example, N,N-dimethyl-N-(n-alkyl)-N-(2-hydroxy-3-chloro-n-propyl)ammonium halides. Furthermore, the cationic protein hydrolysates can also be derivatized still further. Mentioned as typical examples of the cationic protein hydrolysates and derivatives are the products that are commercially available and are known under the INCI names: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

The weight proportion of the protein hydrolysate(s) in terms of the total weight of the hair treatment agents is preferably 0.01 to 5% by weight, more preferably 0.025 to 3% by weight, and in particular 0.05 to 2% by weight.

Irrespective of the source (plant, animal, marine, etc.), protein hydrolysates depending on the degree of hydrolysis include individual amino acids, oligopeptides, and optionally polypeptides.

The hair treatment agents of the invention include with particular preference at least one oligopeptide, which has at least one amino acid sequence Glu-Glu-Glu

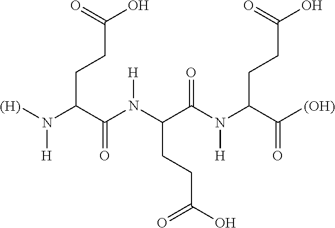

wherein the amino group may be present in a free or protonated form and the carboxy groups in a free or deprotonated form.

Preferred hair treatment agents of the invention are characterized in that, based on their weight, they include 0.0001 to 10% by weight of at least one oligopeptide, which has at least one amino acid sequence Glu-Glu-Glu

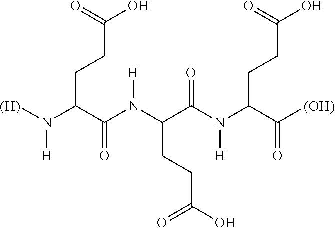

wherein the amino group may be present in a free or protonated form and the carboxy groups in a free or deprotonated form.

In this formula, as in all formulas below, the amino group hydrogen atom in parentheses (H), like the acid function hydroxy group in parentheses (OH), means that the groups in question may be present as such (in which case it is an oligopeptide with the particular number of amino acids as shown (in the present formula 3)) or, however, that the amino acid sequence is present in an oligopeptide that includes still further amino acids (depending on where the further amino acid(s) is/are bound, the components in parentheses in the above formula are replaced by the further amino acid group(s)).

These preferred hair treatment agents of the invention include, based on their weight, 0.0001 to 10% by weight of at least one oligopeptide that has at least one amino acid sequence Glu-Glu-Glu, i.e., at least three consecutive glutamic acids.

Oligopeptides in the context of the present application are amino acid condensation products which are linked acid amide-like by peptide bonds and includes at least 3 and a maximum of 25 amino acids.

In preferred hair treatment agents of the invention, the oligopeptide includes 5 to 15 amino acids, preferably 6 to 13 amino acids, particularly preferably 7 to 12 amino acids, and in particular 8, 9 or 10 amino acids.

Depending on whether additional amino acids are bound to the Glu-Glu-Glu sequence and depending on the type of said amino acids, the molar mass of the oligopeptide present in the agents of the invention may vary. Preferred hair treatment agents used according to the invention are characterized in that the oligopeptide has a molar mass of 650 to 3000 Da, preferably of 750 to 2500 Da, particularly preferably of 850 to 2000 Da, and in particular of 1000 to 1600 Da.

In summary, preferred hair treatment agents are characterized in that the oligopeptide includes 5 to 15 amino acids, preferably 6 to 13 amino acids, particularly preferably 7 to 12 amino acids, and in particular 8, 9, or 10 amino acids and has a molar mass of 650 to 3000 Da, preferably of 750 to 2500 Da, particularly preferably of 850 to 2000 Da, and in particular of 1000 to 1600 Da.

As can be seen from the preferred number of amino acids in the oligopeptides and the preferred molar mass range, oligopeptides consisting not only of the three glutamic acids but also having additional amino acids bound to this sequence are used with preference. These additional amino acids are preferably selected from specific amino acids, whereas specific other representatives are less preferred according to the invention.

It is thus preferred, if the oligopeptides used in the agents of the invention do not include any methionine. It is preferred further, if the oligopeptides used in the agents of the invention do not include any cysteine and/or cystine. It is preferred further, if the oligopeptides used in the agents of the invention do not include any aspartic acid and/or asparagine. It is preferred further, if the oligopeptides used in the agents of the invention do not include any serine and/or threonine.

In contrast, it is preferred, if the oligopeptides used in the agents of the invention include tyrosine. It is preferred further, if the oligopeptides used in the agents of the invention include leucine. It is preferred further, if the oligopeptides used in the agents of the invention include isoleucine. It is preferred further, if the oligopeptides used in the agents of the invention include arginine. It is preferred further, if the oligopeptides used in the agents of the invention include valine.

Particularly preferred oligopeptides or amino acid sequences included in the preferred oligopeptides are described hereafter:

A particularly preferred oligopeptide additionally includes tyrosine, which is bound preferably via its acid function to the Glu-Glu-Glu sequence. Hair treatment agents preferred according to invention are therefore characterized in that the oligopeptide included therein has at least one amino acid sequence Tyr-Glu-Glu-Glu

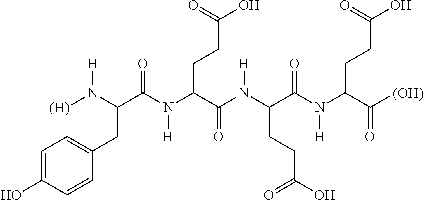

wherein the amino group may be present in a free or protonated form and the carboxy groups in a free or deprotonated form.

A further particularly preferred oligopeptide additionally includes isoleucine, which is bound preferably via its amino function to the Glu-Glu-Glu sequence. Hair treatment agents preferred according to invention are therefore characterized in that the oligopeptide included therein has at least one amino acid sequence Glu-Glu-Glu-Ile

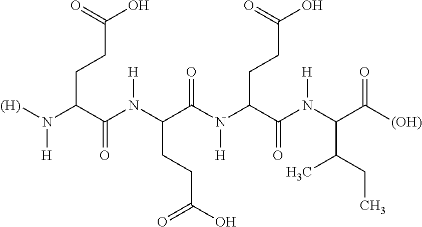

wherein the amino group may be present in a free or protonated form and the carboxy groups in a free or deprotonated form.

Oligopeptides that have both of the aforesaid amino acids (tyrosine and isoleucine) are preferred according to the invention. Particularly preferred in this case are hair treatment agents of the invention in which the oligopeptide included in the hair treatment agent has at least one amino acid sequence Tyr-Glu-Glu-Glu-Ile

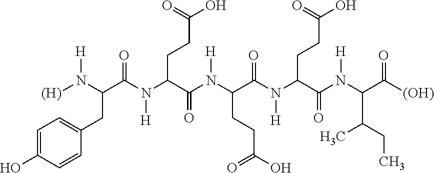

wherein the amino group may be present in a free or protonated form and the carboxy groups in a free or deprotonated form.

Further preferred oligopeptides additionally include arginine, which is preferably present bound to isoleucine. Particularly preferred in this case are hair treatment agents of the invention in which the oligopeptide included in the hair treatment agent has at least one amino acid sequence Tyr-Glu-Glu-Glu-Ile-Arg

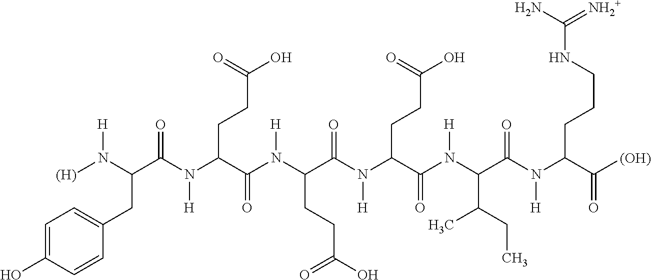

wherein the amino groups may be present in a free or protonated form and the carboxy groups in a free or deprotonated form.

Still further preferred oligopeptides additionally include valine, which is preferably present bound to arginine. Hair treatment agents preferred further according to invention are therefore characterized in that the oligopeptide included in the hair treatment agent has at least one amino acid sequence Tyr-Glu-Glu-Glu-Ile-Arg-Val

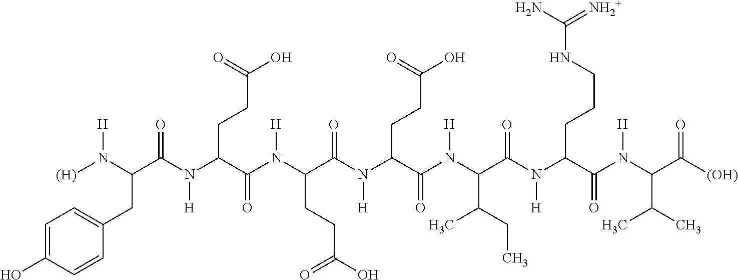

wherein the amino groups may be present in a free or protonated form and the carboxy groups in a free or deprotonated form.

Still further preferred oligopeptides additionally include leucine, which is preferably present bound to valine. Hair treatment agents preferred further according to invention are therefore characterized in that the oligopeptide included in the hair treatment agent has at least one amino acid sequence Tyr-Glu-Glu-Glu-Ile-Arg-Val-Leu

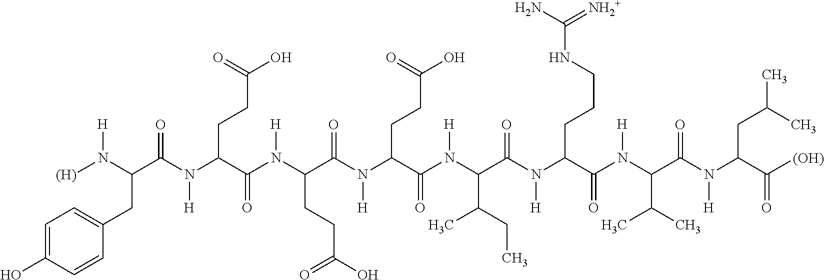

wherein the amino groups may be present in a free or protonated form and the carboxy groups in a free or deprotonated form.

Particularly preferred oligopeptides additionally include leucine, which is preferably present bound to tyrosine. Hair treatment agents preferred further according to invention are therefore characterized in that the oligopeptide included in the hair treatment agent has at least one amino acid sequence Leu-Tyr-Glu-Glu-Glu-Ile-Arg-Val-Leu

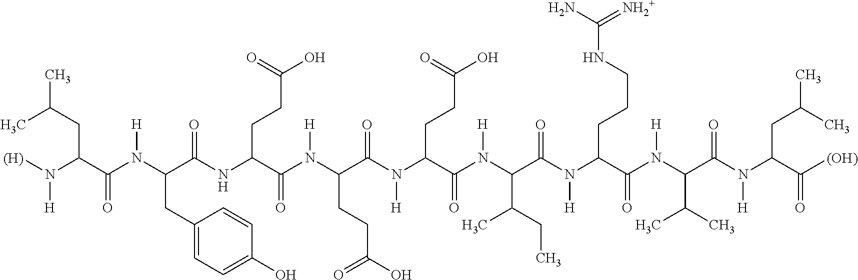

wherein the amino groups may be present in a free or protonated form and the carboxy groups in a free or deprotonated form.

Very particularly preferably, the agents of the invention include at least two oligopeptides that meet the aforesaid criteria, but differ from one another. Thus, for example, the use of hair treatment agents is preferred that include at least two different oligopeptides A and B, both of which include the amino acid sequence Glu-Glu-Glu.

Such different oligopeptides A and B correspond to one another in that they have three consecutive Glu amino acids in their amino acid sequence, but have differences in the amino acids bound before or after these. Different peptides with a partial agreement, which may be much greater than in the aforementioned three amino acids, are preferred.

Thus, further preferred hair treatment agents are characterized in that the hair treatment agent include at least two different oligopeptides A and B, both of which include the amino acid sequence Glu-Glu-Glu-Ile.

Also preferred are hair treatment agents that include at least two different oligopeptides A and B, both of which include the amino acid sequence Tyr-Glu-Glu-Glu.

Even further preferred hair treatment agents are characterized in that they include at least two different oligopeptides A and B, both of which include the amino acid sequence Glu-Glu-Glu-Ile-Arg.

Likewise, even further preferred hair treatment agents are characterized in that the hair treatment agents include at least two different oligopeptides A and B, both of which include the amino acid sequence Tyr-Glu-Glu-Glu-Ile.

Preferred hair treatment agents of the invention are therefore characterized in that the oligopeptide has at least one amino acid sequence Tyr-Glu-Glu-Glu-Ile

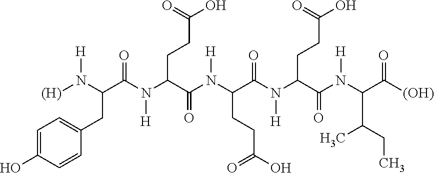

wherein the amino group may be present in a free or protonated form and the carboxy groups in a free or deprotonated form.

Very particularly preferred hair treatment agents are characterized in that the hair treatment agent includes at least two different oligopeptides A and B, both of which include the amino acid sequence Glu-Glu-Glu-Ile-Arg.

Likewise, very particularly preferred hair treatment agents are characterized in that the hair treatment agent includes at least two different oligopeptides A and B, both of which include the amino acid sequence Tyr-Glu-Glu-Glu-Ile-Arg.

Preferably, there is an even greater structural agreement in the oligopeptides. Thus, hair treatment agents that include at least two different oligopeptides A and B, both of which include the amino acid sequence Glu-Glu-Glu-Ile-Arg-Val, are further preferred embodiments of the present invention.

Likewise, preferred embodiments are hair treatment agents that include at least two different oligopeptides A and B, both of which include the amino acid sequence Tyr-Glu-Glu-Glu-Ile-Arg-Val.

Even further preferred hair treatment agents of the invention are characterized in that they include at least two different oligopeptides A and B, both of which include the amino acid sequence Glu-Glu-Glu-Ile-Arg-Val-Leu.

Likewise, still further preferred hair treatment agents of the invention are characterized in that they include at least two different oligopeptides A and B, both of which include the amino acid sequence Tyr-Glu-Glu-Glu-Ile-Arg-Val-Leu.

Preferred hair treatment agents of the invention are therefore characterized in that the oligopeptide has at least one amino acid sequence Tyr-Glu-Glu-Glu-Ile-Arg-Val-Leu

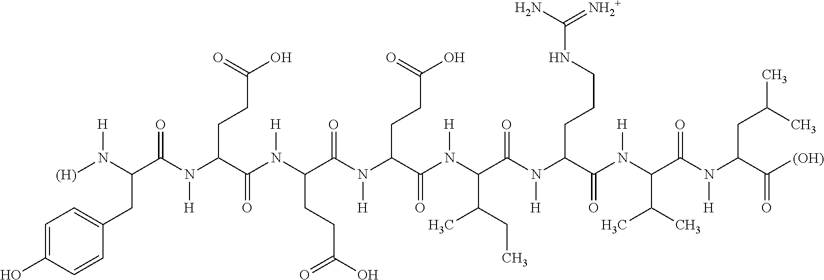

wherein the amino groups may be present in a free or protonated form and the carboxy groups in a free or deprotonated form,

Particularly preferred hair treatment agents of the invention are characterized in that they include at least two different oligopeptides A and B, wherein the oligopeptide A has the amino acid sequence Leu-Tyr-Glu-Glu-Glu-Ile-Arg-Val-Leu

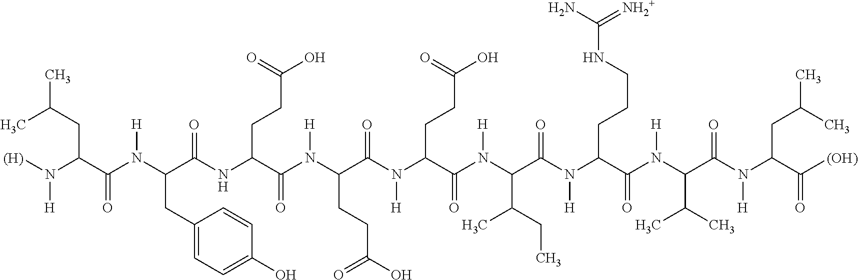

wherein the amino groups may be present in a free or protonated form and the carboxy groups in a free or deprotonated form
and the oligopeptide B has the amino acid sequence Tyr-Glu-Glu-Glu-Ile-Arg-Val-Leu

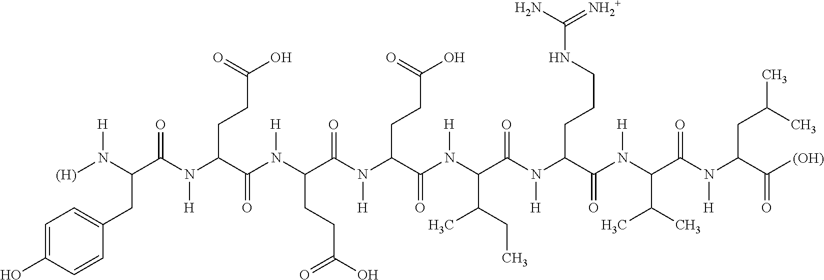

wherein the amino groups may be present in a free or protonated form and the carboxy groups in a free or deprotonated form.

The very particularly preferred hair treatment agents of this latter embodiment include, based on the weight of the agent, 0.00001 to 1% by weight of oligopeptide A and 0.00001 to 1% by weight of oligopeptide B.

The further preferred hair treatment agents of this latter embodiment include, based on the weight of the agent, 0.00005 to 0.1% by weight of oligopeptide A and 0.00005 to 0.1% by weight of oligopeptide B.

The still further preferred hair treatment agents of this latter embodiment include, based on the weight of the agent, 0.0001 to 0.01% by weight of oligopeptide A and 0.0001 to 0.001% by weight of oligopeptide B.

The oligopeptides used in the context of the present invention, which meet the aforesaid conditions, can be obtained advantageously from keratinic materials. It is preferred according to the invention that these oligopeptides are used in large amounts, based on the total peptide content of the agents.

It is very particularly preferred that the greatest possible amount of all keratinic peptides included in the agent of the invention meets the aforesaid conditions.

Preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Glu-Glu-Glu.

Additionally, preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Glu-Glu-Glu-Ile.

Even more preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Tyr-Glu-Glu-Glu.

Particularly preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Tyr-Glu-Glu-Glu-Ile.

Very particularly preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Tyr-Glu-Glu-Glu-Ile-Arg.

Even more particularly preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Tyr-Glu-Glu-Glu-Ile-Arg-Val.

Particularly preferred hair treatment agents of the invention are characterized in that at least 0.1% by weight, preferably at least 0.5% by weight, particularly preferably at least 1% by weight, more preferably at least 2.5% by weight, even more preferably at least 5% by weight, and in particular at least 10% by weight of all keratinic peptides, included in the agent, have the amino acid sequence Tyr-Glu-Glu-Glu-Ile-Arg-Val-Leu.

The aforesaid conditions relate to the total content of peptides, originating from keratinic materials, in the agents of the invention. In addition to the oligopeptides of keratinic origin, naturally further peptides and/or protein hydrolysates can be used, for example, from other native sources. For example, the additional use of wheat protein hydrolysates is preferred.

Suitable vitamins are understood preferably as the following vitamins, provitamins, and vitamin precursors, and derivatives thereof:

Vitamin A: the group of substances designated as vitamin A include retinol (Vitamin A₁) and 3,4-didehydroretinol (vitamin A₂). β-Carotene is the retinol provitamin. Suitable vitamin A components are, for example, vitamin A acid and esters thereof, vitamin A aldehyde, and vitamin A alcohol and esters thereof such as the palmitate and the acetate.

Vitamin B: The vitamin B group or the vitamin B complex includes, inter alia, vitamin B₁ (thiamine); vitamin B₂ (riboflavin); vitamin B₃, where the compounds nicotinic acid and nicotinamide (niacinamide) are often included under this term; vitamin B₅ (pantothenic acid and panthenol). Panthenol is preferably used within the scope of this group. Usable panthenol derivatives are particularly the esters and ethers of panthenol, pantolactone, and cationically derivatized panthenols. Individual representatives are, for example, panthenol triacetate, panthenol monoethyl ether, and the monoacetate thereof, as well as cationic panthenol derivatives; and/or vitamin B₆ (pyridoxine as well as pyridoxamine and pyridoxal).

Vitamin C (ascorbic acid): The use in the form of the palmitic acid ester, glucosides, or phosphates can be preferred. The use in combination with tocopherols can likewise be preferred.

Vitamin E (tocopherols, especially α-tocopherol).

Vitamin F: The term “vitamin F” is conventionally understood to mean essential fatty acids, in particular linoleic acid, linolenic acid, and arachidonic acid.

Vitamin H: Vitamin H is the name for the compound (3aS,4S,6aR)-2-oxohexahydrothienol[3,4-d]-imidazole-4-valeric acid, although the common name of biotin has become accepted.

Vitamins, provitamins, and vitamin precursors from the groups A, B, E, and H are preferred. Preferred in particular are nicotinamide, biotin, pantolactone, and/or panthenol.

The weight proportion of the vitamin(s), vitamin derivative(s), and/or the vitamin precursor(s) in terms of the total weight of the hair treatment agents is preferably 0.001 to 2% by weight, particularly preferably 0.005 to 1% by weight, and in particular 0.01 to 0.5% by weight.

Extracts that can be prepared from all parts of a plant are to be understood as suitable plant extracts. These extracts are typically produced by extraction of the entire plant. It can also be preferred in individual cases, however, to produce the extracts solely from the flowers and/or leaves of the plant. Suitable above all are the extracts from green tea, oak bark, stinging nettle, witch hazel, hops, chamomile, burdock, horsetail, whitethom, lime blossom, lychee, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Boerhavia diffusa roots, Foeniculum vulgaris, and Apium graveolens.

The extracts from green tea, stinging nettle, witch hazel, chamomile, aloe vera, ginseng, Echinacea purpurea, Olea europea, and/or Boerhavia diffusa roots are particularly preferred for use in the compositions of the invention.

Water, alcohols, and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and in a mixture with water. Plant extracts based on water/propylene glycol in the ratio of 1:10 to 10:1 have proven to be particularly suitable.

The plant extracts can be used both in pure and diluted form. If they are used in diluted form, they conventionally include about 2 to 80% by weight of active substance and as the solvent, the extracting agent or mixture of extracting agents used to obtain them.

The plant extracts can be used in the hair treatment agents of the invention, based on the total weight of the agents, preferably in an amount of 0.01 to 10% by weight, more preferably of 0.05 to 7.5% by weight, and in particular of 0.1 to 5% by weight.

Glycerol can be added to the hair cleaning and care agents separately in an amount of up to 10% by weight (based on the total weight of the agent). However, it can also be a component of the aforesaid aqueous-alcoholic carrier.

It was found that the hair treatment agents of the invention are also suitable for use as an anti-dandruff formulation.

The total weight of anti-dandruff agents in terms of the total weight of the hair treatment agents can preferably be 0.01 to 10% by weight, more preferably 0.025 to 7.5% by weight, particularly preferably 0.05 to 5% by weight, and in particular 0.075 to 3% by weight.

Suitable anti-dandruff active agents can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or amica extracts. Climbazole, zinc pyrithione, and piroctone olamine are preferred.

Other active substances, auxiliary substances, and additives that can be included in the hair treatment agents of the invention are, for example: humectants; perfume; UV filters; thickeners, such as gelatin or plant gum, for example, agar-agar, guar gum, alginates, xanthan gum, gum arabic, karaya gum, locust bean gum, flaxseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as, e.g., bentonite, or fully synthetic hydrocolloids such as, e.g., polyvinyl alcohol, Ca, Mg, or Zn soaps; structurants such as maleic acid and lactic acid; dimethyl isosorbide; cyclodextrins; fiber-structure-improving active substances, particularly mono-, di-, and oligosaccharides such as, for example, glucose, galactose, fructose, fruit sugar, and lactose; dyes for coloring the agent; active substances such as bisabolol and/or allantoin; complexing agents such as EDTA, NTA, β-alaninediacetic acid, and phosphonic acids; ceramides, understood to be N-acylsphingosine (fatty acid amides of sphingosine) or synthetic analogs of such lipids (so-called pseudo-ceramides); propellants such as propane-butane mixtures, N₂O, dimethyl ether, CO₂, and air; antioxidants; and/or additional viscosity regulators such as salts (NaCl).

The agents of the invention are preferably so-called rinse-off products, i.e., are rinsed out of the hair again after a specific contact time. This contact time is preferably less than an hour; i.e., the user preferably does not leave the products in the hair until the next hair washing.

A further subject of the present invention is therefore a method for hair treatment in which an agent of the invention is applied to dry or damp hair, is left there for a time period of 30 to 300 seconds, and is rinsed out thereafter.

The agents of the invention lead to a considerably increased strengthening of the internal and extemal hair structure. A further subject of the present invention, therefore, is the use of the agents of the invention for strengthening the hair structure, in particular the internal hair structure.

Strengthening the structure in the context of the invention is to be understood as a reduction in the damage to keratinic fibers caused by a very wide variety of effects. Here, for example, the restoration of the natural strength plays a significant role. Restructured fibers are characterized, for example, by an improved shine, by an improved feel, and by easier combability. In addition, they have an optimized strength and elasticity. A successful strengthening of the structure or restructuring can be determined physically as an increase in the melting point in comparison with damaged fibers.

The statements made about the agents of the invention apply mutatis mutandis in regard to preferred embodiments of the method of the invention and the use of the invention.

### Examples

All quantities are given in % by weight

### Hair Shampoo Series 1

| | 1-1 | 1-2 | 1-3 | 1-4 | 1-5 | 1-6 |
|---|---|---|---|---|---|---|
| C₁₄₋₁₆ alpha-Olefin sulfonate, | 6.0 | 6.0 | 6.0 | 6.0 | 6.0 | 6.0 |
| Na salt (AS) | | | | | | |
| Cocoamidopropyl Betaine | 5.0 | 5.0 | 5.0 | 5.0 | 5.0 | 5.0 |
| (AS) | | | | | | |
| Sodium Methyl Cocoyl | 2.0 | 2.0 | 2.0 | 2.0 | 2.0 | 2.0 |
| Taurate | | | | | | |
| Polyquaternium-10 | 0.3 | 0.3 | 0.3 | 0.3 | 0.3 | 0.3 |
| PEG-7 Glyceryl Cocoate | 1.0 | 1.0 | 1.0 | 1.0 | 1.0 | 1.0 |
| Amodimethicone | 0.8 | 0.8 | 0.8 | 0.8 | 0.8 | 0.8 |
| Hydrolyzed keratin | 0.3 | 0.3 | 0.3 | 0.3 | 0.3 | 0.3 |
| Citric acid | 0.5 | 0.5 | 0.5 | 0.5 | 0.5 | 0.5 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| PEG-120 Methyl Glucose | 0.7 | 0.7 | 0.7 | 0.7 | 0.7 | 0.7 |
| Dioleate | | | | | | |
| o-Cymen-5-ol | 0.1 | 0.1 | 0.1 | 0.1 | 0.1 | 0.1 |
| Ethyl Lauroyl Arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| Hexetidine | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | To 100 |

### Hair Shampoo Series 2

| | 2-1 | 2-2 | 2-3 | 2-4 | 2-5 | 2-6 |
|---|---|---|---|---|---|---|
| C₁₄₋₁₆ alpha-Olefin sulfonate, | 8.0 | 8.0 | 8.0 | 8.0 | 8.0 | 8.0 |
| Na salt (AS) | | | | | | |
| Disodium | 4.0 | 4.0 | 4.0 | 4.0 | 4.0 | 4.0 |
| Cocoamphodiacetate | | | | | | |
| (AS) | | | | | | |
| Sodium Methyl Cocoyl | 4.0 | 4.0 | 4.0 | 4.0 | 4.0 | 4.0 |
| Taurate | | | | | | |
| Cocamide MEA (AS) | 0.3 | 0.3 | 0.3 | 0.3 | 0.3 | 0.3 |
| Guar | 0.1 | 0.1 | 0.1 | 0.1 | 0.1 | 0.1 |
| Hydroxypropyltrimonium | | | | | | |
| Chloride | | | | | | |
| Panthenol | 0.5 | 0.5 | 0.5 | 0.5 | 0.5 | 0.5 |
| Nicotinamide | 0.3 | 0.3 | 0.3 | 0.3 | 0.3 | 0.3 |
| Dimethicone | 0.8 | 0.8 | 0.8 | 0.8 | 0.8 | 0.8 |
| PEG-7 Glyceryl Cocoate | 1.0 | 1.0 | 1.0 | 1.0 | 1.0 | 1.0 |
| Citric acid | 0.5 | 0.5 | 0.5 | 0.5 | 0.5 | 0.5 |
| PEG-120 Methyl Glucose | 0.7 | 0.7 | 0.7 | 0.7 | 0.7 | 0.7 |
| Dioleate | | | | | | |
| o-Cymen-5-ol | 0.06 | 0.06 | 0.06 | 0.06 | 0.06 | 0.06 |
| Ethyl Lauroyl Arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| Hexetidine | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | To 100 |

What is claimed is:

1. A hair treatment agent, comprising:
a) at least one anionic surfactant from the group of alpha-olefin sulfonates,
b) at least one anionic surfactant from the group of taurides,
c) at least one amphoteric surfactant,
d) at least one cationic polymer, and
e) o-cymen-5-ol.

2. The hair treatment agent of claim 1, wherein the at least one anionic surfactant from the group of alpha-olefin sulfonates comprises 0.5% to 20% by weight of the agent.

3. The hair treatment agent of claim 1, wherein the at least one anionic surfactant from the group of alpha-olefin sulfonates comprises 2% to 10% by weight of the agent.

4. The hair treatment agent of claim 1, wherein the at least one anionic surfactant from the group of alpha-olefin sulfonates comprises reaction products of C₁₂-C₁₈ olefins with sulfur trioxide, which consist substantially of alkene sulfonates and hydroxyalkane sulfonates.

5. The hair treatment agent of claim 1, wherein the at least one anionic surfactant from the group of taurides comprises 0.3% to 10% by weight of the agent.

6. The hair treatment agent of claim 1, wherein the at least one anionic surfactant from the group of taurides comprises 0.7% to 5% by weight of the agent.

5. The hair treatment agent of claim 1, wherein the at least one anionic surfactant from the group of taurides comprises taurides of the formula (I),
R¹CON(CH₃)CH₂CH₂SO₃X (I),
where R¹ is a linear or branched alkyl group having 6 to 30 carbon atoms or a linear or branched mono- or polyunsaturated alkenyl group having 6 to 30 carbon atoms, and
X is a counterion, selected from a group consisting of: an alkali metal, alkaline earth metal, and ammonium ion.

6. The hair treatment agent of claim 1, wherein the at least one anionic surfactant from the group of taurides comprises sodium methyl cocoyl taurate.

7. The hair treatment agent of claim 1, wherein the agent comprises no more than 1% by weight of the agent of sulfate surfactant.

8. The hair treatment agent of claim 1, wherein the agent comprises no more than 0.1% by weight of the agent of sulfate surfactant.

9. The hair treatment agent of claim 1, wherein the at least one amphoteric surfactant comprises 0.3% to 10% by weight of the agent.

10. The hair treatment agent of claim 1, wherein the at least one amphoteric surfactant comprises 1% to 5% by weight of the agent.

11. The hair treatment agent of claim 1, wherein the at least one cationic polymer comprises 0.01% to 3% by weight of the agent.

12. The hair treatment agent of claim 1, wherein the at least one cationic polymer comprises 0.15% to 0.8% by weight of the agent.

13. The hair treatment agent of claim 1, wherein the at least one cationic polymer comprises is selected from the group of cationic cellulose polymers, cationic guar derivatives, and mixtures thereof.

14. The hair treatment agent of claim 1, wherein the o-cymen-5-ol comprises 0.001% to 0.4% by weight of the agent.

15. The hair treatment agent of claim 1, wherein the o-cymen-5-ol comprises 0.05% to 0.1% by weight of the agent.

16. The hair treatment agent of claim 1, further comprising 0.0001 to 10% by weight of the agent of at least one oligopeptide with at least one amino acid sequence Glu-Glu-Glu.

17. A hair treatment agent, comprising:
2% to 10% by weight of the agent of at least one anionic surfactant from the group of alpha-olefin sulfonates,
0.7% to 5% by weight of the agent of at least one anionic surfactant from the group of taurides;
1% to 5% by weight of the agent of at least one amphoteric surfactant;
0.15% to 0.8% by weight of the agent of at least one cationic polymer; and
0.05% to 0.1% by weight of the agent of o-cymen-5-ol.

18. The agent of claim 17, wherein the at least one cationic polymer is selected from a group consisting of: cationic cellulose polymers, cationic guar derivatives, and mixtures thereof.

19. A method for hair treatment, comprising:
applying an agent to hair, comprising:
at least one anionic surfactant from the group of alpha-olefin sulfonates,
at least one anionic surfactant from the group of taurides,
at least one amphoteric surfactant,
at least one cationic polymer, and
o-cymen-5-ol; and
rinsing the agent from hair 30 to 300 seconds after application.

20. The method of claim 19, wherein the o-cymen-5-ol comprises 0.001% to 0.4% by weight of the agent
